(19) United States
(12) Patent Application Publication
(10) Pub. No.: US 2019/0027237 A1
(43) Pub. Date: Jan. 24, 2019

(54) BLOCKCHAIN NETWORK FOR SECURE EXCHANGE OF HEALTHCARE INFORMATION

(71) Applicant: Patientory, Inc., Atlanta, GA (US)

(72) Inventor: Chrissa Tanelia McFarlane, Atlanta, GA (US)

(21) Appl. No.: 16/041,469

(22) Filed: Jul. 20, 2018

Related U.S. Application Data
(60) Provisional application No. 62/535,697, filed on Jul. 21, 2017.
(60) Provisional application No. 62/635,076, filed on Feb. 26, 2018.

(51) Int. Cl. G16H 10/60; G06F 17/30; G16H 80/00; H04L 9/30; H04L 9/32

(52) U.S. Cl. CPC G16H 10/60; G06F 17/30283; G06F 17/30206; H04L 9/30; H04L 9/3247; G16H 80/00

(57) ABSTRACT

Disclosed are methods and systems for providing a blockchain network for secure exchange of healthcare information. A system may include a server configured to receive a transaction from a client device via a publicly accessible network, wherein the server authenticates an identity of the client device prior to forwarding the transaction. The system may also include a private blockchain network comprising a data aggregator node configured to receive the transaction forwarded from the server and distribute the transaction to a least a portion of a plurality of processing nodes, wherein each of the plurality of processing nodes executes a respective blockchain client configured to maintain a blockchain having contracts. The private blockchain network may include a forwarder node configured to, in response to detecting an event indicating that the transaction was successfully processed by a respective contract, transact protected health information in a data store separate from the blockchain.

### CROSS-REFERENCE TO RELATED APPLICATIONS

The present application claims the benefit of priority under 35 U.S.C. § 119 from U.S. Provisional Patent Application Ser. No. 62/535,697 entitled “Blockchain Network for Secure Exchange of Healthcare Information,” filed on Jul. 21, 2017, the disclosure of which is hereby incorporated by reference in its entirety for all purposes. The present application is related to U.S. Provisional Patent Application Ser. No. 62/635,076 entitled “Blockchain Network for Secure Exchange of Healthcare Information,” filed on Feb. 26, 2018, the disclosure of which is hereby incorporated by reference in its entirety for all purposes.

### BACKGROUND

As healthcare moves away from transactional care to a more holistic focus on improving patient outcomes, robust and secure electronic medical records (EMR) systems are needed to safely and securely store patient data for various healthcare providers. For example, primary care physicians, caregivers, wellness providers, hospitals, government organizations, and others rely on EMR systems to track patient progress and to comply with evolving healthcare and data privacy regulatory requirements. However, existing EMR systems tend to impose a heavy paperwork or data entry burden on physicians and staff, resulting in accelerated professional burnout and reduced patient quality of care.

Further, existing EMR systems are often proprietary or bespoke solutions for each care provider, resulting in silos of health information that organizations cannot readily or safely share with others. Opportunities are thus lost for improving medical diagnoses and treatments by performing anonymized aggregate data analysis using machine learning or other techniques. Comprehensive individualized healthcare also becomes more difficult as organizations must often resort to laborious manual processes to transfer medical records between different EMR systems. Each transfer of records may also introduce unintended errors and/or the risk of fraud or record falsification. Thus, there is a need to provide improved EMR systems that meet the needs of both healthcare providers and patients.

### BRIEF DESCRIPTION OF THE DRAWINGS

A detailed description will be made with reference to the accompanying drawings:

FIG. 1A illustrates an example architecture for providing a blockchain network for secure exchange of healthcare information.

FIG. 1B is a block diagram illustrating an example client, blockchain node, and server from the architecture of FIG. 1A according to certain aspects of the disclosure.

FIG. 2 is a flowchart illustrating an example data flow between elements of FIGS. 1A and 1B.

FIG. 3 is a diagram illustrating an example user interface for a client device interfacing with a blockchain network of FIG. 1A.

FIG. 4 is a flowchart illustrating an example process for providing a blockchain network for secure exchange of healthcare information.

FIG. 5 is a block diagram illustrating an example computer system with which the clients, servers, and blockchain nodes of FIG. 1A can be implemented.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology may be practiced without these specific details. In some instances, structures and components are shown in block diagram form in order to avoid obscuring the concepts of the subject technology. Like components are labeled with identical element numbers for ease of understanding.

While cryptocurrencies may be the most visible application of blockchain technology, blockchain can support any type of distributed application where centralized authority may be undesirable. By using a distributed ledger of transactions where successive blocks are linked to earlier blocks via a cryptographic function, which can be verified all the way up to the origin block, trust and integrity can be established without using third-party intermediaries. Due to the immutable nature of the blockchain and the significant computational resources an attacker needs to overcome the combined processing power of a blockchain network, users may be assured that their health records are highly resilient against unauthorized modification and tampering.

Thus, blockchain may seem to be an ideal solution for providing secure yet shareable storage of electronic healthcare information. However, there are some significant drawbacks to using a straightforward implementation via a public blockchain. For example, storing sensitive protected health information (PHI) directly on the blockchain risks catastrophic failure if there is a security breach, since the entire blockchain is duplicated and accessible across all public nodes. The storage of PHI on public nodes, even in encrypted form on passive non-mining nodes, may also expose ordinary users to legal liability and regulatory authority. Further, even if a user's private key remains safe, third parties can still obtain significant PHI by observing transactional metadata on the blockchain. Finally, the transactional or cryptocurrency cost to maintain PHI on the blockchain may discourage widespread adoption.

The disclosed system addresses the above problems by providing a private blockchain network for secure exchange of healthcare information. Access to the private blockchain network from a public network such as the Internet is mediated via a server, such as a remote procedure call (RPC) server. The RPC server may authenticate API requests received from clients against an access hierarchy, for example by using asymmetric encryption. One authorized, the API request may be distributed to a portion of active nodes or miners in the private blockchain network for processing into new blocks of the blockchain.

Besides miners, the private blockchain nodes may also include specialized modules such as a forwarder that listens for events on the blockchain corresponding to transactions involving sensitive health information, such as ePHI or PHI covered under Health Insurance Portability and Accountability Act (HIPAA) regulations. The forwarder may interface with a secure HIPAA compliant database to store and retrieve PHI. The secure database may be implemented using a distributed file system. In this manner, sensitive PHI is securely stored in a separate database rather than being directly stored and potentially exposed on the blockchain itself. The blockchain thus stores a minimal subset of information to support smart contracts on the blockchain. Further, since the blockchain is private, potential vectors for security breaches can be minimized.

One or more aspects of the subject technology provide several benefits that improve the functionality and performance of a computer. As discussed above, the blockchain network is private and access is provided through defined API calls that are authenticated using an RPC server validating against an access hierarchy. By enforcing an access hierarchy in such a manner, users and institutions are prevented from unauthorized access and tampering of healthcare information. Thus, the reliability and security of the blockchain network is improved.

Additionally, by using a forwarder that enables PHI to be stored separately from the blockchain, automatic compliance with HIPAA and other regulatory schemes can be readily achieved to reduce operating costs and management overhead. Further, since a size of the blockchain can be correspondingly reduced by storing a minimal subset of information to support smart contracts on the blockchain, a performance of the blockchain network can be improved, for example by reducing processor cycles, power consumption, memory footprint, storage footprint, and network utilization for each node in the blockchain network.

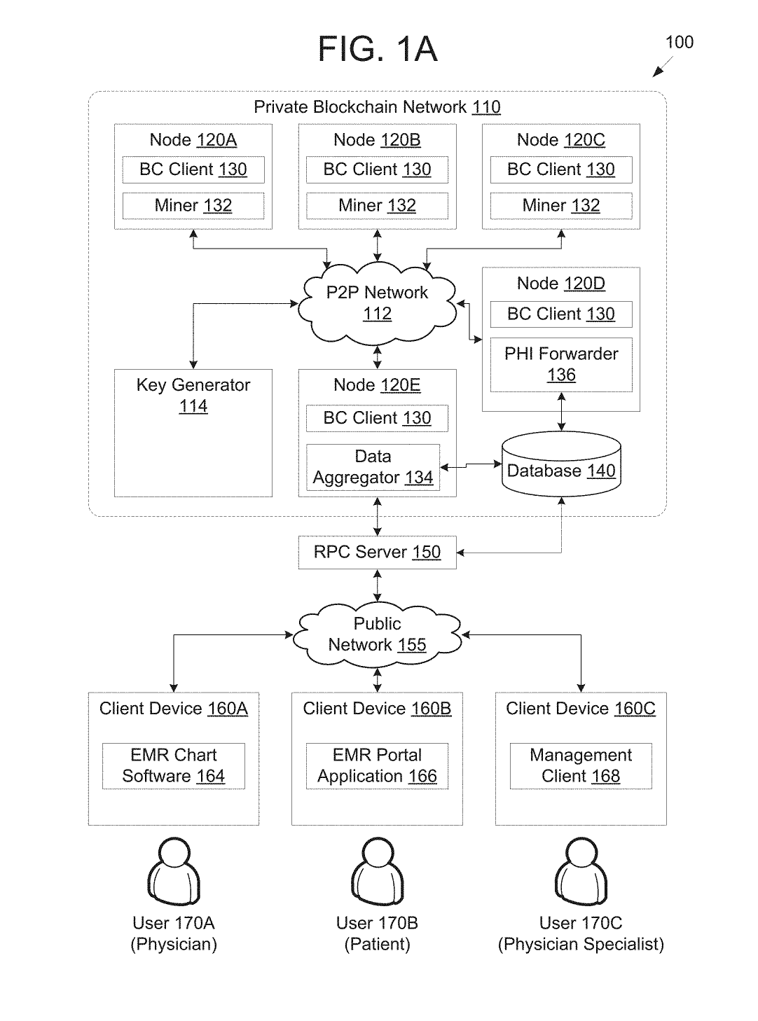

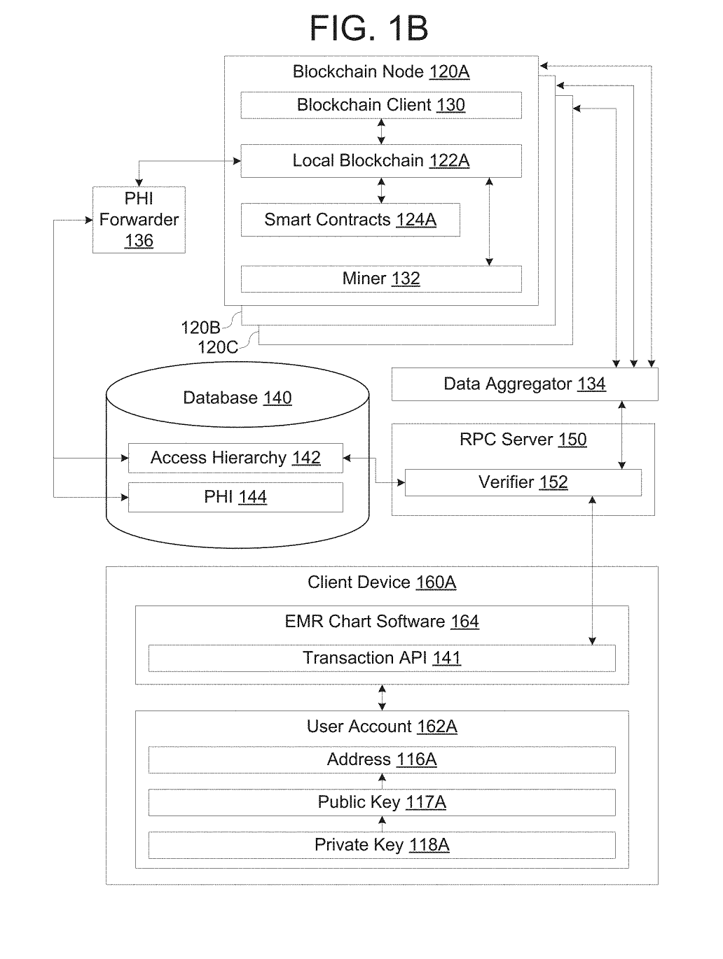

FIG. 1A illustrates an example architecture 100 for providing a private blockchain network 110 for secure exchange of healthcare information. Architecture 100 includes private blockchain network 110, RPC server 150, public network 155, client device 160A, client device 160B, client device 160C, user 170A, user 170B, and user 170C. Private blockchain network 110 includes P2P network 112, key generator 114, node 120A, node 120B, node 120C, node 120D, node 120E, and database 140. Nodes 120A-120C each include blockchain (BC) client 130 and miner 132. Node 120D includes BC client 130 and PHI forwarder 136. Node 120E includes BC client 130 and data aggregator 134. Client device 160A includes EMR chart software 164. Client device 160B includes EMR portal application 166. Client device 160C includes management client 168.

Turning to private blockchain network 110, it can be observed that each of nodes 120A-120E includes BC client 130, which enables each node to at least function as a passive blockchain node storing a local synchronized copy of the blockchain (not specifically shown in FIG. 1A). As shown in FIG. 1A, each node 120A-120E is connected to all the other nodes 120A-120E via P2P network 112, which may be any type of network using any network topology, such as a fully connected network, and may comprise a secure virtual private network (VPN). Nodes 120A-120D may be restricted to communicate solely with other nodes and hosts within private blockchain network 110. A designated node, such as node 120E, includes data aggregator 134 that may be exempt from this restriction to communicate with RPC server 150. While FIG. 1A shows an example with three active nodes and two passive nodes, various quantities and configurations of nodes are possible in private blockchain network 110.

The nodes in private blockchain network 110 may be configured with various different modules. For example, nodes 120A-120C may be active nodes that include miner 132, which may verify pending transactions into new blocks within the blockchain. Passive nodes without miner 132, such as nodes 120D-120E, may synchronize the blockchain via BC client 130 without verifying new blocks. PHI forwarder 136 may identify an event triggered in response to an allowed transaction requesting PHI from or storing PHI into database 140, and may be the sole entity with permissions to perform such transactions with database 140, thus acting as a trusted route. Data aggregator 134 may receive authenticated API requests from RPC server 150 and distribute the API request to one or more active nodes for processing.

Turning to the client side, various users 170A-170C can interact with the health information stored in private blockchain network 110 by using application programs configured to use an API for communication with RPC server 150. As part of an initial registration process, key generator 114 may be utilized to generate private keys for distribution to respective client devices 160A-160C, which may be distributed by optical methods such as two-dimensional barcodes or other means. Alternatively, software executing on the client devices 160A-160C may generate the private keys, such as EMR chart software 164, EMR portal application 166, and management client 168.

As shown in FIG. 1A, various client applications can be developed to meet the different EMR needs of user 170A (Physician), user 170B (Patient) and user 170C (Physician Specialist) while using a common API for communication with RPC server 150. This enables developers to rapidly create new applications and frontends without needing to understand the underlying mechanics of the blockchain system.

Client devices 160A-160C may be any suitable client device including a laptop or desktop computer, a smartphone or tablet, or another computing device that can connect to public network 155, such as the Internet. Client devices 160A-160C may digitally sign API requests using asymmetric or public key encryption, and RPC server 150 may verify the digital signatures based on an access hierarchy stored in database 140 or another location. Once an API request has been authenticated as originating from an approved client device, the API request may be transferred to data aggregator 134 for distribution to active blockchain nodes, as described above.

Having provided an overview for example architecture for providing a blockchain network for secure exchange of healthcare information, it may be instructive to examine the components in more detail. FIG. 1B is a block diagram illustrating an example client device 160A, blockchain node 120A, and RPC server 150 from the architecture of FIG. 1A according to certain aspects of the disclosure. FIG. 1B also includes database 140 and data aggregator 134. Blockchain node 120A includes local blockchain 122A, blockchain client 130, smart contracts 124A, and miner 132. Database 140 includes access hierarchy 142 and PHI 144. RPC server 150 includes verifier 152. Client device 160A includes EMR chart software 164 and user account 162A. EMR chart software 164 includes transaction API 141. User account 162A includes address 116A, public key 117A, and private key 118A. With respect to FIG. 1B, like numbered elements may correspond to similar elements from FIG. 1A.

As described above, each client device, such as client device 160A, may include software that is tailored to the EMR needs of the user associated with the client device. In the case of client device 160A, EMR chart software 164 is provided that is suitable for physicians. Regardless of the particular software interface that is used, a common transaction API 141 may be utilized to communicate with RPC server 150. Further, each client device 160A may be associated with a particular user account, or user account 162A for client device 160A. The private key 118A may be distributed by the blockchain network or generated on the client side, for example by EMR chart software 164, and the remaining components of user account 162A may be derived from private key 118A. Alternatively, rather than using client side software, one or more remote servers may be utilized for providing web-based portals or software as a service/cloud solutions.

Thus, client device 160A may begin by generating an API request using transaction API 141, which is then sent to RPC server 150. Verifier 152 may utilize access hierarchy 142 to authenticate client device 160A. After a successful authentication, the API request is forwarded for processing by one or more active blockchain nodes via data aggregator 134.

For example, access hierarchy 142 may include a one-to-one mapping of user account addresses to access contracts that define the access privileges for each address. Accounts can belong to various class levels including customer or patient level, employee level, or institution level. For example, an access contract to an employee physician may define the patients that the employee physician may manage, and an access contract to an institution may define the employees that it may manage. Appropriate access privileges may be granted, revoked, and updated periodically or on-demand as these relationships evolve over time. Further, since access privileges are defined by access hierarchy 142, backup private keys may be provided for limited emergency access to healthcare information, and lost keys can be readily invalidated and account access can be transferred to new keys, helping to simplify key management.

Blockchain nodes 120B and 120C may include similar components as those shown in blockchain node 120A. Thus, each active blockchain node includes a blockchain client 130, a miner 132, a respective local blockchain 122A-122C and respective smart contracts 124A-124C. The blockchain client 130 may synchronize the local blockchain 122A-122C for each node. Miner 132 may verify transactions or API requests received from data aggregator 134 and add new blocks to local blockchain 122A-122C. Smart contracts 124A-124C may be stored as part of the blockchain.

Since verifying the transactions expends computational resources and since secure storage of PHI 144 on database 140 may also incur a storage and processing cost, the transactions may specify a cryptocurrency fee for completing the transaction. For example, the transactions may specify a fee in Patientory Coin (PTOY) for completing the transactions within private blockchain network 110.

Users may obtain PTOY by various methods. In one example, PTOY may be acquired as part of a pre-sale in an initial coin offering (ICO). In another example, users may acquire PTOY by exchanging another cryptocurrency token, such as ethereum (ETH) or bitcoin (BTC). In yet another example, users may acquire PTOY using third party trading solutions, marketplaces, or exchanges. Once a user has acquired PTOY, the user can spend PTOY to perform transactions on private blockchain network 110.

When transactions (e.g. reads or writes) to PHI 144 are needed, PHI forwarder 136 can detect events on the blockchain and act as a gatekeeper for securely interfacing with PHI 144 stored in database 140. As discussed above, database 140 may be configured to only service requests from PHI forwarder 136 to minimize potential attack vectors.

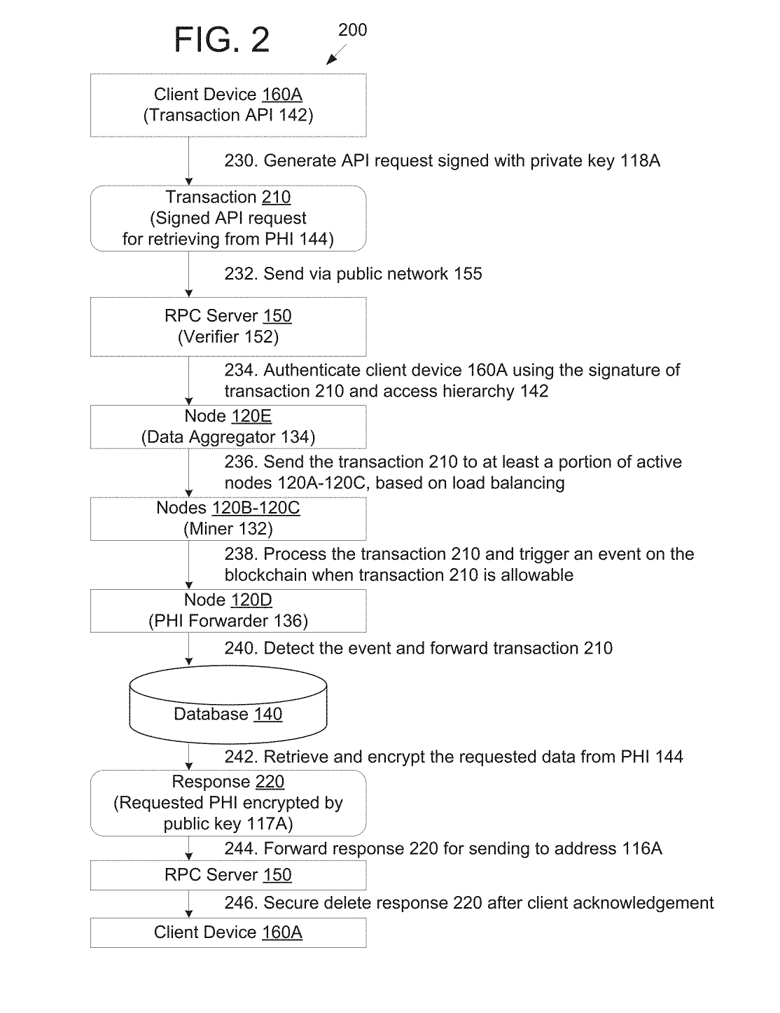

FIG. 2 is a flowchart illustrating an example data flow between elements of FIGS. 1A and 1B. Flowchart 200 includes data aggregator 134, nodes 120A-120C, node 120D, database 140, RPC server 150, client device 160A, transaction 210, response 220, and block 230, 232, 234, 236, 238, 240, 242, 244, and 246. With respect to FIG. 2, like numbered elements may correspond to similar elements from FIGS. 1A and 1B.

At block 230, client device 160 may use transaction API 141 to generate an API request signed with private key 118A. The resulting transaction 210 may be an API request for particular protected health information concerning a particular patient.

At block 232, client device 160A sends the signed API request, or transaction 210, to RPC server 150 via public network 115.

At block 234, RPC server 150 uses verifier 152 to determine whether client device 160A is authenticated for the API request. For example, the signature may be decrypted using a public key of client device 160A stored in access hierarchy 142. If the signature is valid and access hierarchy 142 indicates that user 170A (the physician) is authorized to access the health records associated with the API request (for example, concerning user 170B), then client device 160A may be authenticated. Otherwise, transaction 210 may be denied and flowchart 200 ends early.

At block 236, node 120E uses data aggregator 134 to determine one or more active nodes to send transaction 210. Data aggregator 134 may maintain or periodically request processing load status from each active node and distribute additional transactions based on load balancing all of the available active nodes. For example, if node 120A is already busy working on several transactions whereas node 120B and 120C are relatively idle, then data aggregator 134 may distribute transaction 210 to nodes 120B and 120C, as indicated in the example illustrated in flowchart 200.

At block 238, nodes 120B-120C may use miner 132 to process the transaction 210. Once a minimum number of nodes agree that transaction 210 is valid in the blockchain (e.g. by minimum percentage, simple majority, or other agreement), then an event may be triggered and added into the blockchain to signal the acceptance of transaction 210.

At block 240, node 120D uses PHI forwarder 136 to listen on the blockchain for any new events. The event from block 238 may be detected, and transaction 210 may be forwarded to database 140 via PHI forwarder 136.

At block 242, database 140 retrieves the requested data from PHI 144 according to transaction 210 and encrypts the requested data using the public key 117A of client device 160A to generate response 220.

At block 244, database 140 forwards response 220 via PHI forwarder 136 back to RPC server 150 for sending to address 116A (which is mapped to public key 117A).

At block 246, RPC server 150 sends response 220 to client device 160A associated with address 116A, and securely deletes response 220 from any memory of RPC server 150 after receiving an acknowledgement from client device 160A. In this fashion, RPC server 150 may serve as a conduit for PHI without storing PHI.

While flowchart 200 is illustrated with an example for reading PHI from database 140, a similar process may also be utilized for writing PHI into database 140, with the exception that transaction 210 is encrypted using the public key of database 140.

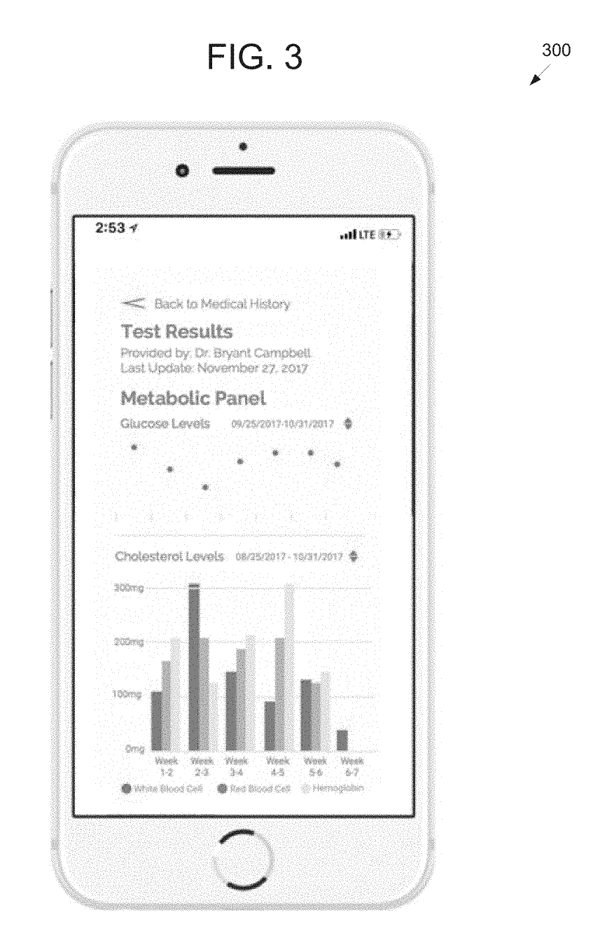

FIG. 3 is a diagram illustrating an example user interface for a client device interfacing with a blockchain network of FIG. 1A. As shown in FIG. 3, user 170B or a patient may access a friendly interface for viewing medical records across different healthcare providers over time, allowing the patient to better track health outcomes and whether treatment plans are providing positive results or may need adjustment. Similarly, user 170A (physician) and user 170C (physician specialist) may access tailored user interfaces via EMR chart software 164 and management client 168. Other stakeholders such as insurance companies, hospital management, and well-being providers may also access user interfaces tailored to their specific needs and processes. Accordingly, each user group can access a user interface that meets the EMR needs of each respective user.

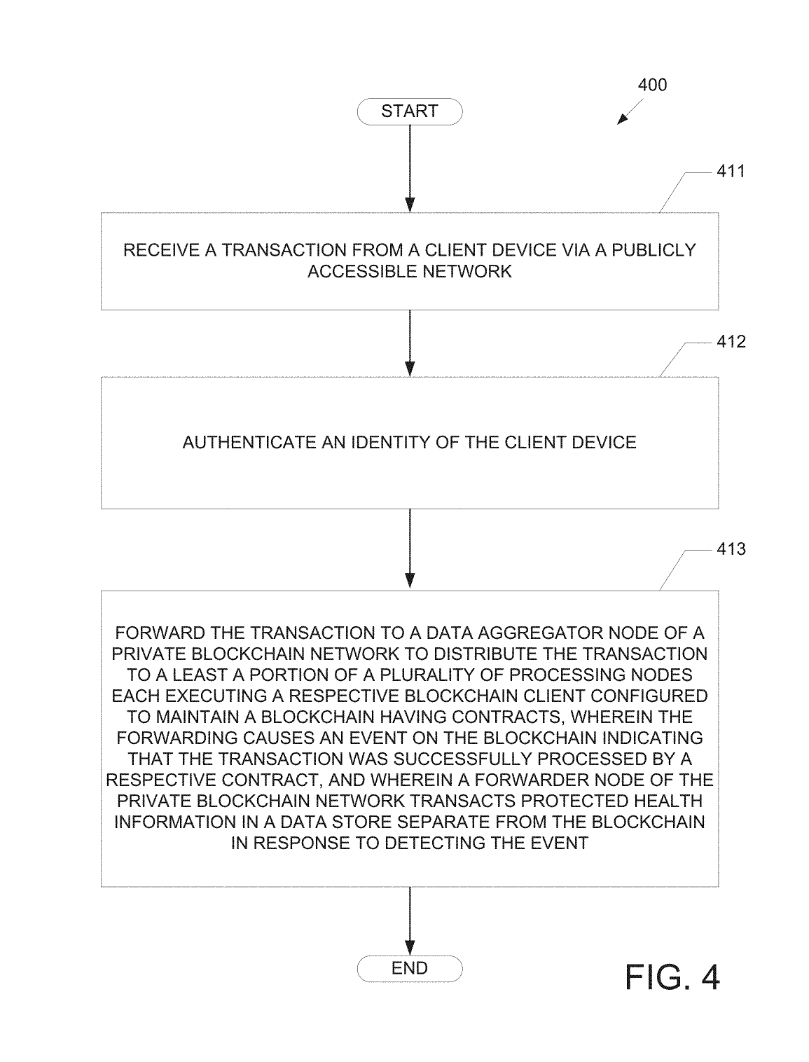

FIG. 4 is a flowchart illustrating an example process 400 for a blockchain network for secure exchange of healthcare information. One or more blocks of FIG. 4 may be executed by a computing system. Similarly, a non-transitory machine-readable medium may include machine-executable instructions thereon that, when executed by a computer or machine, perform the blocks of FIG. 4.

In block 411, referring to FIG. 1A, RPC server 150 receives a transaction from client device 160B via public network 155. As discussed above, user 170B may utilize EMR portal application 166 to access a user interface for reviewing the patient's medical records across various healthcare providers, similar to the interface shown in FIG. 3. For example, user 170B may click on an interface element that allows the user to review bloodwork or metabolic panels. To retrieve the associated protected health information, EMR portal application 166 may first send a transaction to read the associated protected health information.

In block 412, referring to FIG. 1A, RPC server 150 authenticates an identity of client device 160B. For example, the transaction may be signed using a private key of client device 160B. A public key of client device 160B may be stored in access hierarchy 142 and associated with records in PHI 144 associated with user 170B. Thus, after RPC server 150 validates the identity of client device 160B using access hierarchy 142 of database 140, process 400 may proceed to block 413.

In block 413, referring to FIG. 1A, RPC server 150 forwards the transaction to data aggregator 134 of node 120E in private blockchain network 110 to distribute the transaction to at least a portion of nodes 120A-120C each executing BC client 130 configured to maintain a blockchain having contracts. As shown in FIG. 1B, each blockchain node 120A-120C may store a respective local blockchain 122A-122C and smart contracts 124A-124C. Once the miners 132 agree to confirm the transaction, an event may be triggered on the blockchain to indicate that the transaction was successfully processed for a respective contract. PHI forwarder 136 may detect the event and retrieve the requested records from PHI 144, which are then packaged and returned securely to client device 160B, as described in further detail in FIG. 2 above. Once client device 160B receives the requested records, they may be decrypted using a public key of database 140. EMR portal application 166 may then render a user interface based on the requested records, similar to the user interface shown in FIG. 3.

The process 400 can thus securely read or write PHI for any application that is developed using the common transaction API 141, helping to drive adoption and lower development costs. Advantageously, providers do not need to provide their own bespoke solutions or retain information technology staff to plan, deploy, and maintain secure storage and retrieval of protected health information. The described private blockchain network 110 may encourage interoperability across different healthcare participants while simplifying IT deployments, resulting in improved healthcare outcomes for everyone by minimizing burdensome paperwork and management overhead.

### Hardware Overview

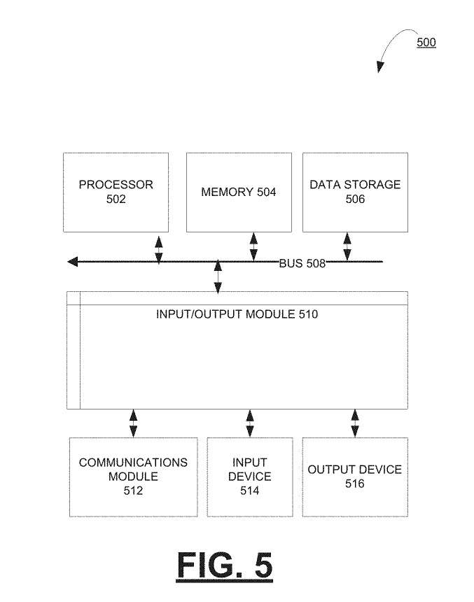

FIG. 5 is a block diagram illustrating an example computer system 500 with which any of nodes 120A-120E, RPC server 150, and client device 160A-160C can be implemented. In certain aspects, the computer system 500 may be implemented using hardware or a combination of software and hardware, either in a dedicated server, or integrated into another entity, or distributed across multiple entities.

Computer system 500 (e.g., nodes 120A-120E, RPC server 150, and client device 160A-160C) includes a bus 508 or other communication mechanism for communicating information, and a processor 502 coupled with bus 508 for processing information. According to one aspect, the computer system 500 can be a cloud computing server of an IaaS that is able to support PaaS and SaaS services. According to one aspect, the computer system 500 is implemented as one or more special-purpose computing devices. The special-purpose computing device may be hard-wired to perform the disclosed techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices, or any other device that incorporates hard-wired and/or program logic to implement the techniques. By way of example, the computer system 500 may be implemented with one or more processors 502. Processor 502 may be a general-purpose microprocessor, a microcontroller, a Digital Signal Processor (DSP), an ASIC, a FPGA, a Programmable Logic Device (PLD), a controller, a state machine, gated logic, discrete hardware components, or any other suitable entity that can perform calculations or other manipulations of information.

Computer system 500 can include, in addition to hardware, code that creates an execution environment for the computer program in question, e.g., code that constitutes processor firmware, a protocol stack, a database management system, an operating system, or a combination of one or more of them stored in an included memory 504, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 508 for storing information and instructions to be executed by processor 502. The processor 502 and the memory 504 can be supplemented by, or incorporated in, special purpose logic circuitry. Expansion memory may also be provided and connected to computer system 500 through input/output module 510, which may include, for example, a SIMM (Single In Line Memory Module) card interface. Such expansion memory may provide extra storage space for computer system 500, or may also store applications or other information for computer system 500. Specifically, expansion memory may include instructions to carry out or supplement the processes described above, and may include secure information also. Thus, for example, expansion memory may be provided as a security module for computer system 500, and may be programmed with instructions that permit secure use of computer system 500. In addition, secure applications may be provided via the SIMM cards, along with additional information, such as placing identifying information on the SIMM card in a non-hackable manner.

The instructions may be stored in the memory 504 and implemented in one or more computer program products, e.g., one or more modules of computer program instructions encoded on a computer readable medium for execution by, or to control the operation of, the computer system 500, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, embeddable languages, and xml-based languages. Memory 504 may also be used for storing temporary variable or other intermediate information during execution of instructions to be executed by processor 502.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 500 further includes a data storage device 506 such as a magnetic disk or optical disk, coupled to bus 508 for storing information and instructions. Computer system 500 may be coupled via input/output module 510 to various devices. The input/output module 510 can be any input/output module. Example input/output modules 510 include data ports such as USB ports. In addition, input/output module 510 may be provided in communication with processor 502, so as to enable near area communication of computer system 500 with other devices. The input/output module 510 may provide, for example, wired communication in some implementations, or wireless communication in other implementations, and multiple interfaces may also be used. The input/output module 510 is configured to connect to a communications module 512. Example communications modules 512 include networking interface cards, such as Ethernet cards and modems.

The components of the system can be interconnected by any form or medium of digital data communication (e.g., a communication network). The communication network (e.g., P2P network 112 and public network 155) can include, for example, any one or more of a personal area network (PAN), a local area network (LAN), a campus area network (CAN), a metropolitan area network (MAN), a wide area network (WAN), a broadband network (BBN), the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

For example, in certain aspects, communications module 512 can provide a two-way data communication coupling to a network link that is connected to a local network. Wireless links and wireless communication may also be implemented. Wireless communication may be provided under various modes or protocols, such as GSM (Global System for Mobile Communications), Short Message Service (SMS), Enhanced Messaging Service (EMS), or Multimedia Messaging Service (MMS) messaging, CDMA (Code Division Multiple Access), Time division multiple access (TDMA), Personal Digital Cellular (PDC), Wideband CDMA, General Packet Radio Service (GPRS), or LTE (Long-Term Evolution), among others. Such communication may occur, for example, through a radio-frequency transceiver. In addition, short-range communication may occur, such as using a BLUETOOTH, WI-FI, or other such transceiver.

In any such implementation, communications module 512 sends and receives electrical, electromagnetic, or optical signals that carry digital data streams representing various types of information. The network link typically provides data communication through one or more networks to other data devices. For example, the network link of the communications module 512 may provide a connection through local network to a host computer or to data equipment operated by an Internet Service Provider (ISP). The ISP in turn provides data communication services through the world wide packet data communication network now commonly referred to as the “Internet.” The local network and Internet both use electrical, electromagnetic, or optical signals that carry digital data streams. The signals through the various networks and the signals on the network link and through communications module 512, which carry the digital data to and from computer system 500, are example forms of transmission media.

Computer system 500 can send messages and receive data, including program code, through the network(s), the network link, and communications module 512. In the Internet example, a server might transmit a requested code for an application program through the Internet, the ISP, the local network, and communications module 512. The received code may be executed by processor 502 as it is received, and/or stored in data storage 506 for later execution.

In certain aspects, the input/output module 510 is configured to connect to a plurality of devices, such as an input device 514 and/or an output device 516. Example input devices 514 include a keyboard and a pointing device, e.g., a mouse or a trackball, by which a user can provide input to the computer system 500. Other kinds of input devices 514 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback, and input from the user can be received in any form, including acoustic, speech, tactile, or brain wave input. Example output devices 516 include display devices, such as an LED (light emitting diode), CRT (cathode ray tube), LCD (liquid crystal display) screen, a TFT LCD (Thin-Film-Transistor Liquid Crystal Display) or an OLED (Organic Light Emitting Diode) display, for displaying information to the user. The output device 516 may comprise appropriate circuitry for driving the output device 516 to present graphical and other information to a user.

According to one aspect of the present disclosure, the client 110A can be implemented using a computer system 500 in response to processor 502 executing one or more sequences of one or more instructions contained in memory 504. Such instructions may be read into memory 504 from another machine-readable medium, such as data storage device 506. Execution of the sequences of instructions contained in main memory 504 causes processor 502 to perform the process steps described herein. One or more processors in a multi-processing arrangement may also be employed to execute the sequences of instructions contained in memory 504. Processor 502 may process the executable instructions and/or data structures by remotely accessing the computer program product, for example by downloading the executable instructions and/or data structures from a remote server through communications module 512 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects, a user of systems and methods as disclosed herein may perform at least some of the steps by accessing a cloud server through a network connection. Further, data files, circuit diagrams, performance specifications, and the like resulting from the disclosure may be stored in a database server in the cloud-computing environment, or may be downloaded to a private storage device from the cloud-computing environment.

Computing system 500 can include clients and servers. A client and server are generally remote from each other and typically interact through a communication network. The relationship of client and server arises by virtue of computer programs running on the respective computers and having a client-server relationship to each other. Computer system 500 can be, for example, and without limitation, a desktop computer, laptop computer, or tablet computer. Computer system 500 can also be embedded in another device, for example, and without limitation, a mobile telephone, a personal digital assistant (PDA), a mobile audio player, a Global Positioning System (GPS) receiver, a video game console, and/or a television set top box.

The term “machine-readable storage medium” or “computer-readable medium” as used herein refers to any medium or media that participates in providing instructions or data to processor 502 for execution. The term “storage medium” as used herein refers to any non-transitory media that store data and/or instructions that cause a machine to operate in a specific fashion. Such a medium may take many forms, including, but not limited to, non-volatile media, volatile media, and transmission media. Non-volatile media include, for example, optical disks, magnetic disks, or flash memory, such as data storage device 506. Volatile media include dynamic memory, such as memory 504. Transmission media include coaxial cables, copper wire, and fiber optics, including the wires that comprise bus 508. Common forms of machine-readable media include, for example, a floppy disk, a flexible disk, a hard disk, magnetic tape, any other magnetic medium, a CD-ROM, a DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire, and fiber optics, including the wires that comprise bus 508. Transmission media can also take the form of acoustic or light waves, such as those generated during radio-wave and infra-red data communications. Furthermore, as used in this specification of this application, the terms “computer,” “server,” “processor,” and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in other one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first, second, and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately, or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

### ASPECTS OF THE INVENTION

In one aspect of the invention, a system is provided, comprising: a server configured to receive a transaction from a client device via a publicly accessible network, wherein the server authenticates an identity of the client device prior to forwarding the transaction; and a private blockchain network comprising: a data aggregator node configured to receive the transaction forwarded from the server and distribute the transaction to a least a portion of a plurality of processing nodes, wherein each of the plurality of processing nodes executes a respective blockchain client configured to maintain a blockchain having contracts; and a forwarder node configured to, in response to detecting an event indicating that the transaction was successfully processed by a respective contract, transact protected health information in a data store separate from the blockchain.

In a further aspect of the invention, the identity of the client device is authenticated by comparing a public key signature of the transaction to an access hierarchy of authorized users.

In a further aspect of the invention, the transaction is provided to the server using an application program interface (API).

In a further aspect of the invention, the server is not directly coupled to the plurality of processing nodes or the forwarder node.

In a further aspect of the invention, the data store is configured to conform to one or more health data privacy rules or regulations.

In a further aspect of the invention, the transaction specifies a cryptocurrency fee for processing the transaction.

In a further aspect of the invention, transacting the protected health information includes at least one of: storing the protected health information into the data store, or retrieving the protected health information from the data store.

In a further aspect of the invention, the protected health information is not stored on non-volatile memory of the server.

In a further aspect of the invention, distributing the transaction to at least the portion of the plurality of processing nodes is based on load balancing the plurality of processing nodes.

In a further aspect of the invention, the respective contract is assigned to the client device in a one-to-one relationship.

In another aspect of the invention, a method is provided comprising: receiving a transaction from a client device via a publicly accessible network; authenticating an identity of the client device; and forwarding the transaction to a data aggregator node of a private blockchain network to distribute the transaction to a least a portion of a plurality of processing nodes each executing a respective blockchain client configured to maintain a blockchain having contracts; wherein the forwarding causes an event on the blockchain indicating that the transaction was successfully processed by a respective contract; and wherein a forwarder node of the private blockchain network transacts protected health information in a data store separate from the blockchain in response to detecting the event.

In a further aspect of the invention, authenticating the identity of the client device comprises comparing a public key signature of the transaction to an access hierarchy of authorized users.

In a further aspect of the invention, the transaction uses an application program interface (API).

In a further aspect of the invention, transacting the protected health information includes at least one of: storing the protected health information into the data store, or retrieving the protected health information from the data store.

In a further aspect of the invention, the protected health information is not stored on non-volatile memory other than the data store.

In a further aspect of the invention, distributing the transaction to at least the portion of the plurality of processing nodes is based on load balancing the plurality of processing nodes.

In a further aspect of the invention, the respective contract is assigned to the client device in a one-to-one relationship.

In another aspect of the invention, a system is provided comprising: means for receiving a transaction from a client device via a publicly accessible network; means for authenticating an identity of the client device prior to forwarding the transaction; means for receiving the transaction forwarded from the server and distributing the transaction to a least a portion of a plurality of processing nodes of a private blockchain network, wherein each of the plurality of processing nodes executes a respective blockchain client configured to maintain a blockchain having contracts; and means for transacting protected health information in a data store separate from the blockchain in response to detecting an event indicating that the transaction was successfully processed by a respective contract.

In a further aspect of the invention, the data store is configured to conform to one or more health data privacy rules or regulations.

In a further aspect of the invention, the means for transacting protected health information is configured to perform at least one of: storing the protected health information into the data store, or retrieving the protected health information from the data store.

What is claimed is:

1. A system, comprising:
a server configured to receive a transaction from a client device via a publicly accessible network, wherein the server authenticates an identity of the client device prior to forwarding the transaction; and
a private blockchain network comprising:
a data aggregator node configured to receive the transaction forwarded from the server and distribute the transaction to a least a portion of a plurality of processing nodes, wherein each of the plurality of processing nodes executes a respective blockchain client configured to maintain a blockchain having contracts; and
a forwarder node configured to, in response to detecting an event indicating that the transaction was successfully processed by a respective contract, transact protected health information in a data store separate from the blockchain.

2. The system of claim 1, wherein the identity of the client device is authenticated by comparing a public key signature of the transaction to an access hierarchy of authorized users.

3. The system of claim 1, wherein the transaction is provided to the server using an application program interface (API).

4. The system of claim 1, wherein the server is not directly coupled to the plurality of processing nodes or the forwarder node.

5. The system of claim 1, wherein the data store is configured to conform to one or more health data privacy rules or regulations.

6. The system of claim 1, wherein the transaction specifies a cryptocurrency fee for processing the transaction.

7. The system of claim 1, wherein transacting the protected health information includes at least one of: storing the protected health information into the data store, or retrieving the protected health information from the data store.

8. The system of claim 1, wherein the protected health information is not stored on non-volatile memory of the server.

9. The system of claim 1, wherein distributing the transaction to at least the portion of the plurality of processing nodes is based on load balancing the plurality of processing nodes.

10. The system of claim 1, wherein the respective contract is assigned to the client device in a one-to-one relationship.

11. A method comprising:
receiving a transaction from a client device via a publicly accessible network;
authenticating an identity of the client device; and
forwarding the transaction to a data aggregator node of a private blockchain network to distribute the transaction to a least a portion of a plurality of processing nodes each executing a respective blockchain client configured to maintain a blockchain having contracts;
wherein the forwarding causes an event on the blockchain indicating that the transaction was successfully processed by a respective contract; and
wherein a forwarder node of the private blockchain network transacts protected health information in a data store separate from the blockchain in response to detecting the event.

12. The method of claim 11, wherein authenticating the identity of the client device comprises comparing a public key signature of the transaction to an access hierarchy of authorized users.

13. The method of claim 11, wherein the transaction uses an application program interface (API).

14. The method of claim 11, wherein transacting the protected health information includes at least one of: storing the protected health information into the data store, or retrieving the protected health information from the data store.

15. The method of claim 11, wherein the protected health information is not stored on non-volatile memory other than the data store.

16. The method of claim 11, wherein distributing the transaction to at least the portion of the plurality of processing nodes is based on load balancing the plurality of processing nodes.

17. The method of claim 11, wherein the respective contract is assigned to the client device in a one-to-one relationship.

18. A system comprising:
means for receiving a transaction from a client device via a publicly accessible network;
means for authenticating an identity of the client device prior to forwarding the transaction;
means for receiving the transaction forwarded from the server and distributing the transaction to a least a portion of a plurality of processing nodes of a private blockchain network, wherein each of the plurality of processing nodes executes a respective blockchain client configured to maintain a blockchain having contracts; and
means for transacting protected health information in a data store separate from the blockchain in response to detecting an event indicating that the transaction was successfully processed by a respective contract.

19. The system of claim 18, wherein the data store is configured to conform to one or more health data privacy rules or regulations.

20. The system of claim 18, wherein the means for transacting protected health information is configured to perform at least one of: storing the protected health information into the data store, or retrieving the protected health information from the data store.
